(19) United States
(12) Patent Application Publication
(10) Pub. No.: US 2017/0238895 A1
(43) Pub. Date: Aug. 24, 2017

(54) METHOD AND APPARATUS FOR RESPIRATION-CORRELATED COMPUTED TOMOGRAPHY IMAGING

(71) Applicant: Siemens Healthcare GmbH, Erlangen (DE)

(72) Inventor: Christian Hofmann, Erlangen (DE)

(73) Assignee: Siemens Healthcare GmbH, Erlangen (DE)

(21) Appl. No.: 15/435,589

(22) Filed: Feb. 17, 2017

(30) Foreign Application Priority Data
Feb. 19, 2016 (DE) 102016202605.4

(51) Int. Cl. A61B 6/00; A61B 5/08; A61B 6/03

(52) U.S. Cl. CPC A61B 6/541; A61B 6/032; A61B 5/0816

(57) ABSTRACT

In a method and apparatus for respiration-correlated computed tomography imaging, a patient-specific breathing curve is recorded and is evaluated online, and a CT scan, providing a number of raw images of a region of interest of a patient, is controlled synchronously with the patient-specific breathing curve according to the results of the online evaluation.

### BACKGROUND OF THE INVENTION

Field of the Invention

The present invention concerns a method for computed tomography imaging wherein the acquisition of raw image data is implemented dependent on the respiration of the patient. The invention also concerns a computed tomography apparatus for implementing such a method.

Description of the Prior Art

Computed tomography (CT) is an X-ray technique for obtaining cross-sectional images of a three-dimensional object by analyzing 2D raw (projection) images. Compared to a conventional fluoroscopic X-ray in which only comparatively coarse structures and bones can be identified, CT imaging captures even soft tissue with minimal contrast difference and in detail. CT images can be generated in two dimensions (2D-CT) or in three dimensions (3D-CT). In three-dimensional CT, absorption profiles of the region of interest are created from many directions and the volume structure of a scanned region is reconstructed therefrom (3D-CT volumes).

Reconstructed 3D-CT volumes are now routinely used in radiotherapy planning, e.g. for lung and abdominal carcinomas. The aim of radiotherapy is to destroy malignant tissue by means of ionizing radiation while causing as little damage as possible to healthy tissue. Before commencing any radiotherapy, radiation planning tailored to the malignant tissue is performed.

When obtaining raw images for reconstructing the 3D-CT volumes, it is necessary to take the breathing of the patient under examination into account for this purpose. The tissue structures moved during imaging produce undesirable artifacts in the reconstructed image. In order to minimize these artifacts, ideally only raw images that correlate with a specific breathing phase are used for reconstruction. For optimum reconstruction, a sufficient number of raw images must therefore be available for each breathing phase. In the case of a scanning procedure in which a circumferential scan is carried out to obtain a large number of raw images at different longitudinal or Z-positions, the scan should therefore ideally cover at least one complete breathing cycle of the patient. In this way the movement of tumors and tissue at risk over the entirety of the breathing cycle can be delimited, and a planning target volume (PTV) that is as small as possible can be obtained. In order to be able to assign the raw images to a specific breathing phase, so-called respiration-correlated imaging techniques are known, wherein a breathing curve, used as a breathing surrogate, is plotted synchronously with image capture, and is stored with the raw images.

External breathing surrogates that are used, for example, as the basis for reconstructing the 3D-CT volumes are customarily produced using suitable sensors. Such sensors are, for example, spirometers or expansion belts. A spirometer measures the volume of air inspired and expired by the patient, whereas a belt with chest expansion transducers measures changes in thoracic or abdominal circumference. Camera systems that record the movements of reflecting markers on the patient's chest are also suitable as sensors.

However, the accuracy of the volume images obtained by such conventional respiration-correlated reconstruction techniques depends on the patient breathing regularly and reproducibly during raw data acquisition, i.e. optimally with a constant breathing rate and amplitude. However, this is not true of every patient. Irregularities in breathing rate and amplitude result in inconsistent and incomplete raw data. In particular, raw images that are correlated to the same breathing phase consequently show differences in anatomy. This in turn results in undesirable artifacts in the final reconstructions.

### SUMMARY OF THE INVENTION

An object of the invention is to provide a method for respiration-correlated computed tomography imaging that enables multidimensional images to be reconstructed with minimal artifacts even in the case of breathing irregularities.

A further object of the invention is to provide an apparatus with which multidimensional images can be reconstructed with minimal artifacts even in the case of breathing irregularities.

The method-based object of the invention is inventively achieved by a method for respiration-correlated computed tomography imaging, wherein a patient-specific breathing curve is recorded and evaluated online, and a CT scan providing a number of raw images is controlled synchronously with the patient-specific breathing curve depending on the results of the online evaluation.

In a first step, the invention is based on the fact that, during raw data acquisition, i.e. during the scan, a patient's respiration signal is currently only registered in parallel time with the scan. Although breathing irregularities are detected, an immediate reaction to such irregularities during the scan, such as changing the scanning rate, the scan time or the number of projections obtained per scan, is not possible.

In a second step, the invention is based on the consideration that each position of a region of interest of a patient must be scanned for at least the duration of a complete breathing cycle in order to acquire a complete respiration-correlated 4D-CT data set. However, in the event of irregularities such as g. a change in the respiratory rate, breathing pauses or coughing, it can occur that data acquisition at a first scan position is incomplete and scanning continues at the subsequent position of the region of interest even before the breathing cycle has ended.

The invention recognizes in a third step that, even in the event of irregularities in the patient's breathing, acquisition of a complete respiration-correlated 4D-CT data set is possible if a patient-specific breathing curve recorded during the scan is evaluated online, and the scan is controlled synchronously with the patient-specific breathing curve depending on the results of the online evaluation. In other words, the execution of the scan is directly influenced by the simultaneous respiration of the patient. The controlling of the scan consequently tracks the time-parallel breathing curve of the patient. The term “breathing curve” denotes a time characteristic of measured values that correlates with the patient's breathing, i.e. a respiration signal, and therefore includes the breathing surrogates that would be acquired using sensors. The term “online evaluation” means in particular real-time analysis.

Online evaluation of the patient-specific breathing curve in real time makes it possible to react to breathing rate fluctuations or to irregularities in amplitude. In particular, the time of raw data acquisition is adjusted according to the currently recorded breathing curve. For this purpose, for each position of the region of interest, the duration of the scan is extended according to the currently recorded breathing curve until sufficient raw data or raw images are present for a sufficient number of breathing phases showing a sufficient quality of respiration. The event of irregularities in the breathing curve, the duration of the scan is extended until at least one complete breathing cycle has been acquired for a particular position of the region of interest. This means that, depending on the desired phase resolution, a breathing cycle that is complete in this sense is composed of a sufficient number of phase sections in which there is regular respiration of sufficient quality. This prevents, in a scan of an examination position, raw data relating to specific breathing phases not being available or being only of insufficient diagnostic value.

In other words, by ensuring immediate reaction to breathing-related changes during the scan, online evaluation enables reproducible 3D images to be produced on the basis of consistent raw data and with fewer 4D artifacts in the CT reconstructions. According to the invention, the breathing curve obtained during the scan is also recorded and stored along with the raw data, i.e. raw images. Assigning a breathing phase to the raw images allows respiration-correlated reconstruction of the 3D image data.

The breathing curve reproduces the patient-specific breathing pattern, i.e. the periodically repeated inspiration and expiration of the patient. In an embodiment of the invention, the breathing curve is used to determine a start time and an end time for the scan online, and the scan is commenced at the start time and terminated at the end time. The duration of the scan is therefore directly linked to the current breathing rate, i.e. the current duration of a patient's breathing process. A change in the breathing rate is directly detected and results in an appropriate change in the duration of the scan. Both the start time and the end time are expediently selected as time values of significant points on the breathing curve. One such significant point is, for example, an extreme or inflection point in the breathing curve. If the attainment of such a significant point in the breathing curve is detected, the corresponding time value is taken as the start time and a scan is commenced. The scan is then carried out over a period of time Δt until the next significant point is detected. The time value of detection of the next significant point is set as the end time and the scan is terminated. In an alternative variant, a number of and/or different significant points on the breathing curve are evaluated and/or counted, and the start time and end time are inferred from the sequence of a number of significant points and are used to control the duration of the scan.

The significant points are preferably selected such that the period of time Δt between the start time and the end time corresponds at least to the duration of a complete breathing cycle, so that a scan encompasses all the phases of a complete breath including inspiration and expiration. In the case of uniform and normal respiration of the patient, the period of time Δt of the scan between the start time and the end time therefore encompasses a complete breathing cycle regardless of the current breathing rate. In the event of irregularities, such as when the breathing suddenly becomes shallow, possibly no significant point is detected in an actual breathing cycle as the end time in the patient-specific breathing curve evaluated online. In this case the period of time Δt between the start time and the end time of the scan is extended to a detected significant point in the breathing curve, thereby considerably improving the consistency of the raw data acquired. The quality of breathing is simultaneously assessed via the evaluation and detection of significant points in the breathing curve.

Expediently a first scan is carried out in parallel time with a first breathing cycle at a fixed position in the longitudinal direction of the patient and upon completion of the first scan, a second scan is performed in parallel time with a second breathing cycle at a position downstream of the position in the longitudinal direction of the patient. In other words, at each longitudinal position of the region of interest corresponding to the desired resolution or the detector width, a complete scan contingent on the online evaluation is carried out. Accordingly, the CT imaging system is preferably operated in a sequential scan mode in which consecutive positions zₖ to zₖ₊ₓ of a region of interest of a patient are scanned one after the other. For this purpose the patient to be scanned is placed on a positioning device, usually a table. The table is moved to a fixed position zₖ and at this position zₖ, a scan is carried out, the duration of which is in this case dependent on the evaluation of the breathing curve acquired in parallel time. Usually a gantry of the computed tomography system rotates around the region of interest of the patient at the position zₖ, so the patient is penetrated by X-rays from different directions so that a number of corresponding raw images are obtained in the form of projections. The rotation time of the gantry for a single rotation or rather a single revolution around the patient is usually about 0.5 seconds. Depending on the CT system used, typically between 1000 and 2000 projections are obtained during each revolution of the gantry. In the case of an adult's typical breathing cycle duration of 6 seconds, the gantry therefore rotates around the patient about 12 times in order to capture a complete breathing cycle, so that some 12000 to 24000 projections are obtained within a breathing cycle.

The start time is preferably determined as a time value of a breathing curve extreme corresponding to a first inhalation maximum, and wherein the end time is determined as a time value of a subsequent breathing curve extreme corresponding to a second inhalation maximum. A breathing surrogate typically has one significant extreme per cycle, usually a maximum amplitude, which corresponds to the inhalation maximum within the breathing cycle. By detecting this extreme or rather the associated point in time, the breathing rate can therefore be obtained from the current breathing curve. The scan is therefore started by the inhalation maximum of a first breath (i.e. by the instant of its occurrence) and stopped by the inhalation maximum of a subsequent breath (i.e. by the instant of its occurrence). Scanning then moves to a position zₖ₊₁ subsequent to the first position zₖ where the procedure is repeated. The period of time Δt between two consecutive inhalation maxima is at least one breathing cycle. In the event of breathing-related irregularities, a number of breathing cycles are encompassed by the period Δt. This is the case if the patient's breathing is at times so shallow that no inhalation maximum is determined as an extreme in a breathing cycle.

In an embodiment of the invention, the start time and/or the end time is/are only determined as a time value of the, or each, extreme if the absolute value of the amplitude of the respective extreme exceeds a predefined value. This value is determined prior to the scan on the basis of the breathing pattern or rather the breathing curve of the gently and evenly breathing patient. The scan is only started or stopped if an extreme is identified and, as an additional condition, the absolute value of the amplitude of the extreme or of the breathing curve has exceeded the setpoint value. This ensures that the scan is started in a regular breathing cycle and stopped in a regular breathing cycle. Accordingly, the scan is continued until consistent projection data for a complete breathing cycle has been obtained. Irregularities in the amplitude of the respiration signal, e.g. during coughing or shallow breathing, etc., do not cause the scan to be aborted.

It is further preferred that the specifically stored patient-specific breathing curve is evaluated offline upon completion of the scan. If the patient unexpectedly coughs during scanning or the quality of the breathing curve is very poor in general, this can affect the amplitude quality of a breathing cycle. This can mean that no extrema, i.e. no start time and/or no end time of a scan, are found at a position zₖ and therefore that no complete breathing cycle is scanned. Likewise, the breathing curve may lack amplitude information for reconstructing a complete breathing cycle. Offline evaluation of the breathing curve enables such irregularities to be detected and suitable action to be taken. Such action can include repeating a scan at a particular position, selecting particular raw images for reconstruction or correcting the phase assignment of the raw images. Offline evaluation can be performed after each scan carried out at a particular position if order to re-scan if required without longitudinal table feed being necessary.

Alternatively, using offline evaluation, breathing curve irregularities occurring during the scan can be identified in correlation to a position in the longitudinal direction of the patient. This makes it possible to selectively repeat scans at a specific position even after the examination is complete. In the event of irregularities in the breathing curve being detected at a specific position, a re-scan is therefore performed at this position as a suitable and expedient measure.

For evaluation of the breathing curve acquired, in a preferred variant a representative breathing curve is predefined and/or learned from a plurality of prior patient-specific breathing curves, wherein the patient-specific breathing curve is evaluated online on the basis of the representative breathing curve. This and the preferred further developments explained below are independently inventive per se, wherein the method of evaluation is particularly and preferably suitable for online evaluation of a breathing curve in order to thereby control the scan for computed tomography imaging. The corresponding features of such an imaging technique are immaterial for the method of evaluation by means of a representative breathing curve.

A representative breathing curve of the patient is preferably learned or inferred using a number of the patient's breathing curves, which are acquired prior to the actual examination, i.e. in advance. A current or specifically present breathing curve is expediently evaluated by direct comparison with the representative breathing curve. This makes it possible to predict specific time instants in the current breathing curve, e.g. the reaching of the maximum inhalation time. The currently acquired and ongoing breathing curve is therefore rapidly evaluated and in real time in order to control the scan during imaging.

The start and end time for the scan is advantageously determined online from the patient-specific breathing curve, in particular on the basis of the representative breathing curve. In particular, these times are determined on the basis of the attainment of corresponding extrema in the current breathing curve by comparison with the representative breathing curve, for which purpose particular conditions for the respective attainment can be derived from the representative breathing curve. Such conditions are, for example, the time characteristic of the amplitude as such, specific sections in the breathing curve which correspond to specific breathing phases, gradient values or the time characteristic of the gradient, i.e. the rate of change in the breathing curve, etc.

In another preferred variant, tuples of amplitude and time derivative are formed from values of the representative breathing curve for evaluation, wherein the coordinates of a center are determined from the tuples, wherein the tuples in respect of the center are converted into polar coordinates, wherein a particular angle is assigned to a representative breathing curve extreme corresponding to an inhalation maximum, wherein values of the patient-specific breathing curve are converted online into polar coordinates in respect of the center determined from the representative breathing curve, wherein a current angle is determined therefrom in each case, and wherein by comparing the currently determined angle with the particular angle the time value of an extreme is determined online. The coordinates of the center are preferably determined as coordinates of the geometric center of the tuples of amplitude and derivative.

The advantage of the variant described above is that the attainment of an extreme in the currently acquired breathing curve is indicated directly via the angle evaluated. The particular angle indicating the attainment of the extreme is not attained in the evaluation of a breathing curve if the amplitude of a current breathing cycle is less than the amplitude of the representative breathing curve. In this case, the center of the representative breathing curve is geometrically outside the characteristic of the current breathing curve, i.e. of the current breathing cycle, described by the value pair, i.e. tuple of amplitude and derivative, so that the angles evaluated in respect of the center do not describe a complete cycle. The same applies accordingly to the respective derivative values. In other words, breathing cycles having irregularities in characteristic and in amplitude are deemed to be irregular by the specified evaluation algorithm, as no extreme is indicated. However, the scan is therefore continued in the desired manner until consistent raw data or raw images for all the phase sections of a complete breathing cycle has been obtained.

The further object of the invention is inventively achieved by an apparatus for respiration-correlated computed tomography imaging, having a CT scanner for performing a scan of a region of interest of a patient and providing a plurality of raw images, a sensor for acquiring a patient-specific breathing curve, and a control computer designed to carry out the above described method.

The CT scanner expediently has a gantry having an X-ray source and a detector. The gantry is designed to rotate about a patient in order to carry out a scan. For this purpose the patient is positioned on a table which can be moved in the longitudinal direction of the patient. As part of the examination process, the patient's region of interest is positioned accordingly with respect to the gantry. For a sequential scanning mode, the table is then moved to different, consecutive positions zₖ, wherein a scan of the region of interest is performed at each position zₖ.

A spirometer or a belt with chest expansion transducers is expediently used as a sensor for acquiring the breathing curve or breathing surrogates. To record the breathing curve, a spirometer measures the volume of air inspired and expired by the patient, whereas a belt with chest expansion transducers measures changes in thoracic or abdominal circumference. In an alternative variant, for further expediency a camera system, via which movements of in particular reflecting markers positioned on the patient's chest are observed, is used as a sensor.

### BRIEF DESCRIPTION OF THE DRAWINGS

FIG. 1 shows an example of a patient-specific breathing curve.

FIG. 2 shows a representative breathing curve determined from the real breathing curve.

FIG. 3 shows real breathing curves and the representative breathing curve in an amplitude/speed graph.

FIG. 4 shows an apparatus for respiration-correlated computed tomography imaging, comprising a CT scanner and a sensor for capturing breathing curves.

### DESCRIPTION OF THE PREFERRED EMBODIMENTS

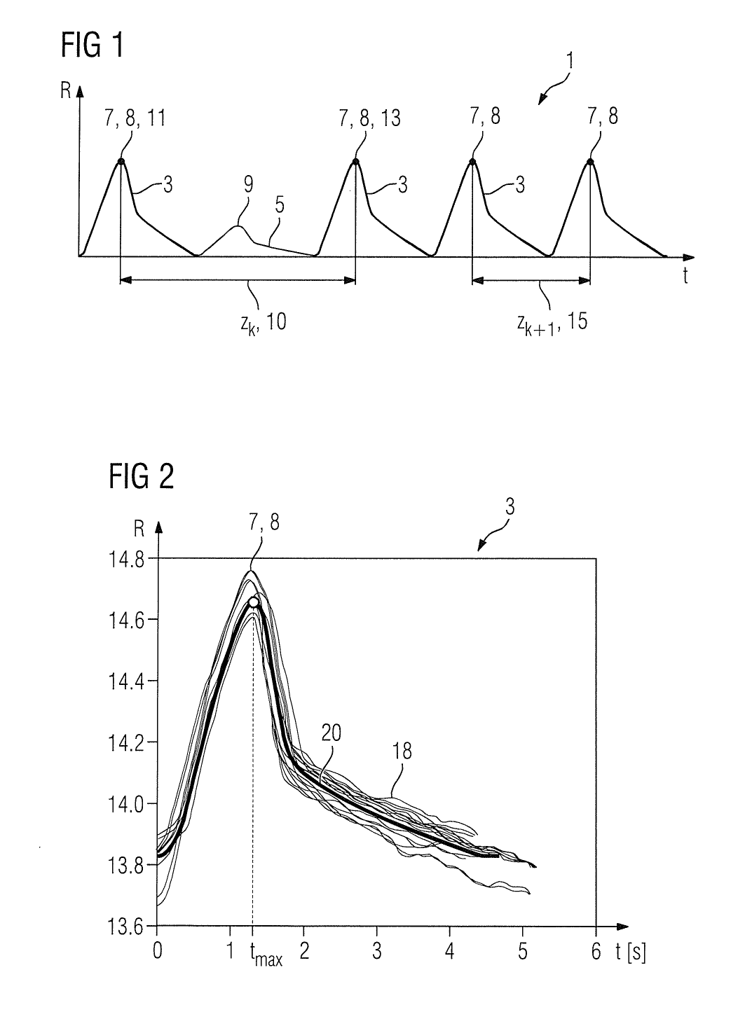

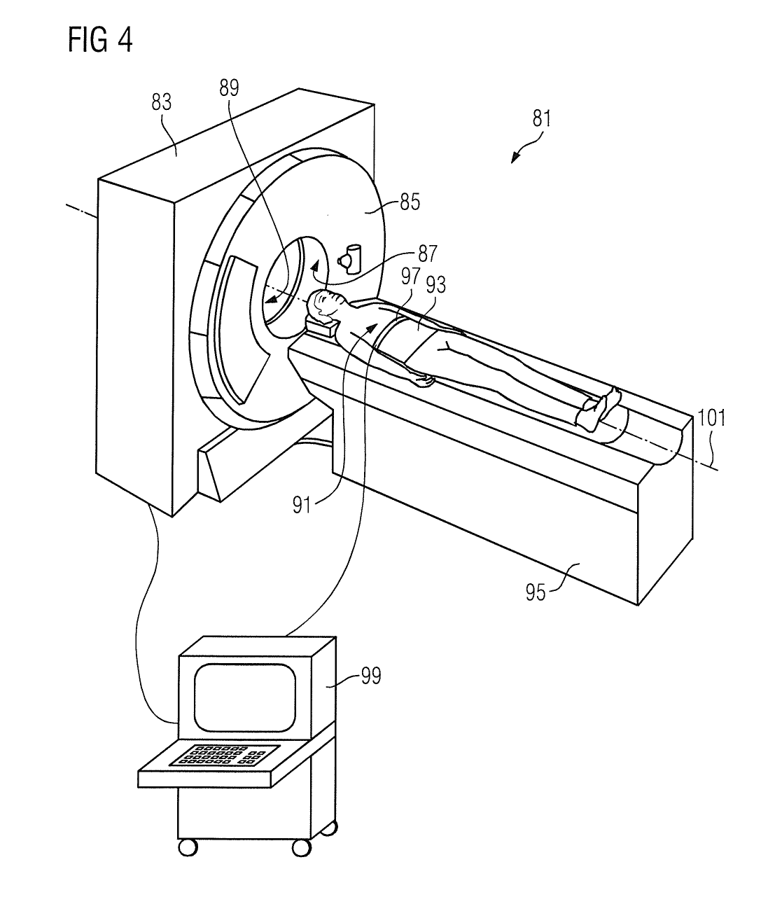

FIG. 1 shows a typical breathing curve 1 of a patient. The breathing curve 1 is measured/recorded as a breathing surrogate using an external sensor, e.g. a spirometer or a belt with chest expansion transducers. To control a CT scan providing a plurality of raw images of a region of interest of the patient, the acquired patient-specific breathing curve is evaluated online. The scan is then controlled according to the results of the online evaluation. A corresponding apparatus for this purpose and the corresponding apparatus components are shown in FIG. 4.

Controlling the scan via the online evaluation of the synchronously acquired breathing curve ensures in particular that, at a position zₖ of the region of interest of the patient, sufficient consistent raw data for a complete breathing cycle is recorded in order to be able to reconstruct 3D images of the region of interest without artifacts caused by breathing irregularities.

The breathing curve 1 shown in FIG. 1 in this case comprises five breathing cycles 3. A second breathing cycle 5 thereof exhibits irregularities in its amplitude. This may be due to the measurement data or to a changed respiration of the patient. To perform a scan, the current breathing curve 1 is evaluated online after points 7 of maximum inhalation. In this case these are points of maximum amplitude, i.e. extrema 8 along the breathing curve 1. The breathing cycle 5 also shows an extreme 9. However, this is at a significantly reduced amplitude compared to the other extremes 8.

After the start of the examination process, a first scan 10 shall be performed at a first fixed position zₖ in the longitudinal direction of the patient. For this purpose the occurrence of the first extreme 8 is determined from the online evaluation of the breathing curve 1 as the start time 11. On reaching or detecting the start time 11, the first scan 10 begins. While the first scan is being carried out, with a plurality of raw images being captured from different directions, the breathing curve 1 continues to be synchronously evaluated, i.e. in parallel time. The next extreme 9 of the second breathing cycle 5 is not detected as a relevant inhalation maximum, as its amplitude value is small compared to a predefined setpoint value. It is only in a further breathing cycle 3 that the online evaluation identifies another extreme 8 in the breathing curve 1, as the amplitude is sufficiently high. Only this extreme 8 of the further breathing cycle 3 is evaluated by the online evaluation as an end time 13, the detection or attainment of which terminates the first scan 10.

It can be seen that the first scan 10 extends over a period of two breathing cycles 3. The second, irregular breathing cycle 5 is identified as irregular. Consequently, the scan 10 is continued until sufficient raw data have been acquired over each phase of a regular breathing cycle 2. Raw data are used that are not from an inhalation phase of the irregular, second breathing cycle 5, but are from an inhalation phase of the next regular breathing cycle 3, in order to reconstruct a 3D image at the position zₖ.

On completion of the first scan 10, a second scan 15 is carried out with the patient's positioning changed (table feed control), at a position zₖ₊₁. In contrast to the first scan 10, the simultaneously further evaluated breathing curve 1 here shows breathing irregularities. On detection or attainment of a point 7 of maximum inhalation, i.e. the extreme 8 of a breathing cycle 3, the second scan 15 is started. On detection or attainment of a point 7 of maximum inhalation, i.e. the extreme 8 of the subsequent breathing cycle 3, the second scan 15 is terminated. The amplitude values at the two extrema 8 exceed a predefined setpoint value in each case. The second scan 15 lasts for a duration of one breathing cycle.

The method is then continued, moving the patient along until a scan has been performed at all the desired positions zₖ. It will be immediately apparent that, using the method specified, the duration of a respective scan is directly linked to the breathing rate. A longer or shorter breathing cycle results in a corresponding adjustment to the scan duration.

In an advantageous variant, after a scan has been performed or when scanning of all the positions is complete, offline evaluation of the acquired breathing curves 1 is carried out. If the breathing curve if found to have irregularities or to be of very poor quality, the corresponding position zₖ is re-scanned, in particular prioritized according to the severity of the irregularity. An irregularity resulting in re-scanning is, in particular, missing amplitude information in the breathing curve, which information is necessary for phase-selected reconstruction of the 3D image.

FIG. 2 shows a representative breathing curve 20 (continuous bold line) averaged or learned from a number of actually measured previous breathing curves 18 of a patient over the duration of a breathing cycle. At a position tₘₐₓ, the representative breathing curve 20 has a point 7 of maximum inhalation, i.e. its extreme 8, as is typically the case for regular breathing curves 3 as shown in FIG. 1. The representative breathing curve 20 is specifically obtained in advance prior to performing an examination, e.g. based on a number of recorded breathing curves 18 of the patient. It is also possible to predefine the representative breathing curve 20 in a patient-specific manner on the basis of empirical values, for which purpose appropriate databases are accessed.

The representative breathing curve 20 is used for online evaluation of a breathing curve 1 according to FIG. 1 in order to control a scan for CT imaging during an examination or treatment of the patient.

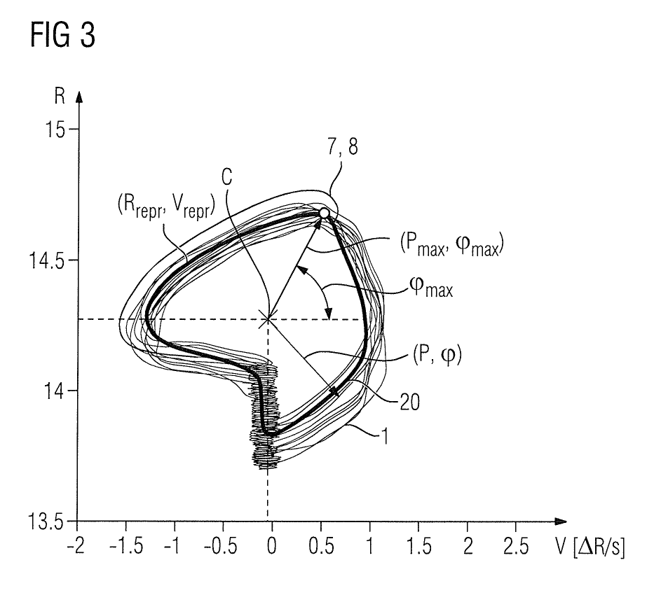

To that end FIG. 3 shows the representative breathing curve 20 for a duration of one breathing cycle and a family of currently recorded breathing curves 1 in an amplitude/rate graph. The respective amplitude values R are here plotted against the rate values V determined as a time derivative. Because of the periodicity of the signal, the plot of the value pairs or tuples (Rᵣₑₚᵣ, Vᵣₑₚᵣ) of the representative breathing curve 20 produces, for a breathing cycle, the rotation of a characteristic circle that is angle-dependent in its radius. Corresponding online evaluation of a currently recorded breathing curve 1 produces a curved path rotating along this circle, or with few deviations.

From the tuples (Rᵣₑₚᵣ, Vᵣₑₚᵣ) of the representative breathing curve 20, a geometric (mid-point) or center C predefined via amplitude and rate values is determined, in respect of which the tuples of the breathing curves 1, 20 are converted into polar coordinates, i.e. distance P and phase angle φ. The center C is marked with its coordinates (C_{R}, C_{V}) in FIG. 3. In particular, there is produced for the representative breathing curve 20 a characteristic angle φₘₐₓ that is assigned to the extreme 8 or more precisely the instant of maximum inhalation 8. In the graph according to FIG. 3, the corresponding tuple is marked with (Pₘₐₓ, φₘₐₓ). For online evaluation of a current breathing curve 1 in which the phase angle φ(t) is determined continuously or in time-discrete increments, the resulting unambiguous criterion for establishing the occurrence of the corresponding extreme is φ(tᵢ)<φₘₐₓ and φ(tᵢ₊₁)>φₘₐₓ. In this case the extreme has been attained or exceeded, and the corresponding time value can be used as the start time or end time for commencing/terminating a scan as described above.

Depending on the selection of the center C, in the case of an irregular breathing cycle with low amplitude, no phase angles are determined in the region of φₘₐₓ by the online evaluation described if the corresponding breathing curve according to FIG. 3, for example, lies below the center C. In this case, the scan is continued until a regular abort criterion has been established on the basis of a regular breathing cycle.

Actually measured breathing curves 1 of the patient are used at regular intervals to adjust the representative breathing curve 20. Shifts, particularly baseline drift, in the respiration signals are therefore reacted to. Also the representative breathing curve 20 is continuously matched to the actual breathing of the patient. Depending on the particular representative breathing curve 20, the calculation of the center C is adjusted accordingly.

FIG. 4 shows an apparatus 81 for respiration-correlated computed tomography imaging. The apparatus 81 includes a CT scanner 83 having a rotatable gantry 85 comprising a fan beam X-ray source 87 and a circular segmented, flat panel detector 89. To perform a scan of a region of interest 91 of a patient 93, said scan providing a plurality of raw images, the apparatus has a sensor 97 for recording a patient-specific breathing curve 1, and a control computer 99 designed to carry out the method as claimed in one of the preceding claims.

As preparation for e.g. radiotherapy, the computed tomography scanner 83 is used to perform a CT scan of a region of interest 91 of the patient 93. Consecutive positions are successively scanned. To carry out the examination, i.e. imaging, the table 95 on which the patient is positioned is fed to a first fixed position zₖ. The gantry 85 rotates about the patient at this position zₖ until consistent raw data for at least one complete breathing cycle has been obtained. The duration of a scan at a position of the patient 93 is determined by means of online evaluation of a synchronously recorded breathing curve of the patient 93. The breathing curve itself is acquired as a breathing surrogate during the CT scan by means of a sensor 97. A belt with chest expansion transducers, for example, is used as a sensor 97. Alternatively, a spirometer is used. The table 95 on which the patient is positioned is then moved along the longitudinal direction of the patient 101 to a next fixed position zₖ₊₁ and a new scan is performed.

Each scan is controlled at the respective position zₖ depending on the results of the online-evaluated patient-specific breathing curve. An appropriately implemented control unit 99 is used for this purpose. The control computer 99 is designed to carry out the method for controlling a scan on the basis of online evaluation of the current breathing curve as described above.

Although modifications and changes may be suggested by those skilled in the art, it is the intention of the Applicant to embody within the patent warranted hereon all changes and modifications as reasonably and properly come within the scope of the Applicant's contribution to the art.

1. A method for respiration-correlated computed tomography (CT) imaging, comprising:
while a patient is situated in a CT scanner, obtaining a breathing curve, representing respiration of the patient, providing said breathing curve to a processor, and evaluating said breathing curve online in real time in said processor;
while said breathing curve is evaluated online, operating the CT scanner to implement a CT scan to obtain a plurality of sets of raw CT data controlled synchronously with the breathing curve according to results of the online evaluation, and thereby obtaining respiration-correlated sets of raw CT data; and
making the sets of respiration-correlated raw CT data available in electronic form, as at least one data file, from said processor.

2. A method as claimed in claim 1 comprising, in said processor, determining a start time and an end time for said scan from said breathing curve, and controlling said CT scan from said processor to begin at said start time and to terminate at said end time.

3. A method as claimed in claim 2 comprising determining said start time and said end time so that at least one breathing cycle of the patient occurs therebetween.

4. A method as claimed in claim 3 comprising, from said processor, operating said CT scanner to execute a first CT scan of the patient in parallel time with a first breathing cycle of the patient, with the patient at a fixed position in a longitudinal direction of the patient and, upon completion of said first CT scan, operating said CT scanner from said processor to execute a second CT scan in parallel time with another breathing cycle of the patient, with the patient at a position longitudinally downstream of said position of the patient during said first CT scan.

5. A method as claimed in claim 2 comprising determining said start time as a time of occurrence of an extreme value of said breathing curve corresponding to a first inhalation maximum, and determining said end time as a time of occurrence of a subsequent extreme value of said breathing curve, corresponding to a second inhalation maximum.

6. A method as claimed in claim 5 comprising determining at least one of said start time or said end time as corresponding to the time of occurrence of the respective extreme value only if an absolute value of an amplitude of the respective extreme value exceeds a predetermined set value.

7. A method as claimed in claim 1 comprising additionally evaluating said breathing curve of the patient offline after completion of said CT scan.

8. A method as claimed in claim 7 comprising, in said offline evaluation, identifying irregularities in said breathing curve that occurred during said CT scan, and correlating said irregularities with a position within the patient along a longitudinal direction of the patient.

9. A method as claimed in claim 8 comprising, for any position along said longitudinal direction of the patient at which one of said irregularities is identified, re-scanning the patient at that position.

10. A method as claimed in claim 1 comprising determining a representative breathing curve for the patient as a predefined breathing curve or a learned breathing curve that is learned based on a plurality of breathing curves of the patient, and evaluating the breathing curve online during said CT scan dependent on said representative breathing curve.

11. A method as claimed in claim 10 comprising determining a start time and an end time for said CT scan online from the breathing curve obtained from the patient relative to the representative breathing curve.

12. A method as claimed in claim 11 comprising determining the start time and the end time as respective times of occurrence of extreme values in said representative breathing curve.

13. A method as claimed in claim 12 comprising, in said processor:
determining tuples of amplitude and derivatives with respect to time from values of the representative breathing curve;
determining coordinates of a center from said tuples and converting the tuples, with respect to said center, into polar coordinates, and assigning a respective angle to each extreme value of the representative breathing curve in said polar coordinates, said extreme values corresponding to respective inhalation maximums;
concerting values of the breathing curve obtained from the patient during said CT scan into polar coordinates with respect to said center determined from said representative breathing curve; and
determining a curve polar angle in said polar coordinates for each value of the breathing curve of the patient obtained during said CT scan, and determining a time of occurrence of said extreme values online by comparing the currently determined angle of the breathing curve of the patient obtained during the CT scan with the angle assigned to an extreme value of said representative breathing curve in said polar coordinates.

14. A computed tomography (CT) apparatus comprising:
a CT scanner adapted to receive a patient therein;
a breathing detector configured to generate a breathing curve that represents respiration of the patient in the CT scanner; and
a control computer configured to operate the CT scanner to implement a CT scan of the patient, said control computer being supplied with said breathing curve online during said CT scan and being configured to evaluate said breathing curve in real time and to control said CT scan in real time dependent on the evaluation of said breathing curve.
